(19) United States
(12) Patent Application Publication
(10) Pub. No.: US 2025/0275726 A1
(43) Pub. Date: Sep. 4, 2025

(54) METHOD FOR TRAINING A MACHINE LEARNING MODEL, MONITORING DEVICE, AND MONITORING METHOD

(71) Applicant: TERUMO KABUSHIKI KAISHA, Tokyo (JP)

(72) Inventors: Tomonori HATTA, Hadano Kanagawa (JP); Takayuki UCHIDA, Hiratsuka Kanagawa (JP); Yuta MIYAOKA, Meguro Tokyo (JP)

(21) Appl. No.: 19/209,756

(22) Filed: May 15, 2025

Related U.S. Application Data
(63) Continuation of application No. PCT/JP2023/041388, filed on Nov. 17, 2023.

(30) Foreign Application Priority Data
Nov. 18, 2022 (JP) 2022-185046

(51) Int. Cl. A61B 5/00; A61B 5/0205; A61B 5/021; A61B 5/024; A61B 5/029; A61B 5/145; G16H 40/67; G16H 50/50

(52) U.S. Cl. CPC A61B 5/7267; A61B 5/0205; A61B 5/742; G16H 40/67; G16H 50/50; A61B 5/021; A61B 5/024; A61B 5/029; A61B 5/14546; A61B 5/6852

(57) ABSTRACT

A method for training a machine learning model executed to assess a condition of a patient with heart failure, includes generating training data by performing for each patient acquiring a first target value based on an invasive test and a first parameter value based on a non-invasive test at a first time, and acquiring a second target value based on the invasive test and a second parameter value based on the non-invasive test at a second time, deriving a target change ratio based on the first and second target values, deriving a parameter change ratio based on the first and second parameter values, and storing the change ratios as the training data, and training a machine learning model with the training data such that a target change ratio is generated in response to an input of an actual parameter change ratio derived for a patient with heart failure.

### CROSS-REFERENCE TO RELATED APPLICATION

This application is a continuation of International Patent Application No. PCT/JP2023/041388 filed Nov. 17, 2023, which is based upon and claims the benefit of priority from Japanese Patent Application No. 2022-185046, filed Nov. 18, 2022, the entire contents of which are incorporated herein by reference.

### BACKGROUND

### Technical Field

Embodiments described herein relate to a method for training a machine learning model, a monitoring device, and a monitoring method.

### Related Art

Heart disease, together with cancer and stroke, is collectively referred to as the three major diseases. These three major diseases are the leading causes of death among Japanese people. Heart disease is a generic term for diseases caused by structural or functional abnormalities of the heart. Examples of heart disease include heart failure, ischemic heart disease, valvular heart disease, cardiomyopathy, arrhythmia, and congenital heart disease.

Heart failure is a condition in which the pumping function of the heart decreases due to an organic or functional disorder of the heart, leading to a decrease in cardiac output, peripheral circulatory failure, and congestion of the lungs and the systemic venous system. Heart failure is classified as acute or chronic depending on the rate of progression. In order to prevent acute exacerbations for a patient who has completed acute treatment and discharged from a hospital and for a patient who has been diagnosed with chronic heart failure, it is required to periodically check their disease state and evaluate the cardiac function. Therefore, those patients need to visit the hospital regularly, but frequent visits burden both the patient and the healthcare system. Consequently, there is a need to assess the disease state and evaluate the cardiac function using biological information measured by heart failure patients in their homes.

A device and a method for non-invasive left ventricular end-diastolic pressure (LVEDP) measurement are available for home monitoring. However, such a device and method do not consider patients' individual differences in their normal range of biological parameters, which may make it difficult to accurately access disease status and evaluate cardiac function.

### SUMMARY

Embodiments of this disclosure provide a method for training a machine learning model, a monitoring device, and a monitoring method.

A method for training a machine learning model for assessing a condition of a remotely monitored patient with heart failure, comprises: generating training data by performing for each of a plurality of patients with heart failure: acquiring at least a first target value based on a result of an invasive test performed at a first time and at least a first parameter value based on a result of a non-invasive test performed at the first time, and then acquiring at least a second target value based on a result of the invasive test performed at a second time and at least a second parameter value based on a result of the non-invasive test performed at the second time, the first and second target values including at least one of: an intracardiac pressure, a value of brain natriuretic peptide (BNP), a value of N-terminal pro-B-type natriuretic peptide (NT-proBNP), a uric acid level, an inferior arterial diameter, and a ventricular ejection fraction, and the first and second parameter values including at least one of: a pre-ejection period (PEP), a left ventricular ejection time (LVET), a diastolic blood pressure, a systolic blood pressure, a maximum rate of rise of a pulse pressure waveform, a pressure difference between a rising start point and a dicrotic notch of a peripheral pulse pressure waveform, a pulse wave increase coefficient, a heart rate, an isovolumetric systolic time, a pulse wave velocity, and a systolic time, deriving a target value change ratio based on the first and second target values, deriving a parameter value change ratio based on the first and second parameter values, and storing the target value change ratio in association with the parameter value change ratio as the training data; and training a machine learning model with the generated training data such that a target value change ratio is generated in response to an input of an actual parameter value change ratio derived for a remotely monitored patient with heart failure.

In one aspect of the present application, it is possible to generate training data and optimize a machine learning model using the training data, and it is possible to accurately estimate the intracardiac pressure using the optimized machine learning model.

### BRIEF DESCRIPTION OF DRAWINGS

FIG. 1 is an explanatory diagram illustrating a configuration example of a medical monitoring system.

FIG. 2 is a block diagram illustrating a hardware configuration example of a monitoring server.

FIG. 3 is a block diagram illustrating a hardware configuration example of an in-hospital terminal.

FIG. 4 is a block diagram illustrating a hardware configuration example of a user terminal.

FIG. 5 is an explanatory diagram illustrating an example of a patient database (DB).

FIG. 6 is an explanatory diagram illustrating an example of a feature amount DB.

FIG. 7 is an explanatory diagram illustrating an example of a home feature amount DB.

FIG. 8 is an explanatory diagram illustrating an example of a threshold DB.

FIG. 9 is an explanatory diagram illustrating an example of a result DB.

FIG. 10 is an explanatory diagram illustrating an example of a coefficient DB.

FIG. 11 is an explanatory diagram illustrating an example of a point sequence DB.

FIG. 12 is an explanatory diagram illustrating an example of a prescription DB.

FIG. 13 is an explanatory diagram illustrating an example of a drug-taking status DB.

FIG. 14 is a flowchart illustrating a procedure example of estimation model generation processing.

FIG. 15 is a flowchart illustrating a procedure example of training data creation processing.

FIG. 16 is an explanatory diagram illustrating an example of constructing a data set.

FIG. 17 is a flowchart illustrating a procedure example of learning processing.

FIG. 18 is an explanatory diagram illustrating an example of an estimation model.

FIG. 19 is a flowchart illustrating a procedure example of collection processing.

FIG. 20 is a flowchart illustrating a procedure example of estimation processing.

FIG. 21 is an explanatory diagram illustrating an example of a result list screen.

FIG. 22 is an explanatory diagram illustrating an example of constructing a data set.

FIG. 23 is an explanatory diagram illustrating an example of a fitting model.

FIG. 24 is a flowchart illustrating a procedure example of result screen generation processing.

FIG. 25 is an explanatory diagram illustrating an example of a nurse result screen.

FIG. 26 is an explanatory diagram illustrating an example of a doctor result screen.

FIG. 27 is an explanatory diagram illustrating an example of a patient result screen.

FIG. 28 is an explanatory diagram illustrating an example of a patient trend display screen.

FIG. 29 is an explanatory diagram illustrating an example of a patient notification screen.

### DETAILED DESCRIPTION

### First Embodiment

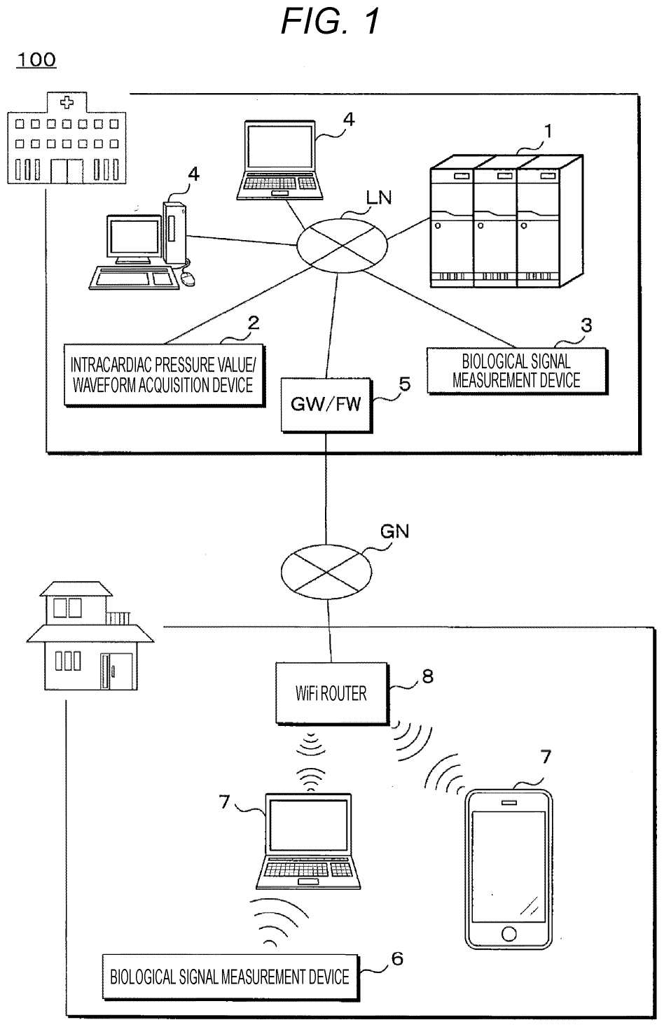

Hereinafter, embodiments will be described with reference to the drawings. FIG. 1 is an explanatory diagram illustrating a configuration example of a medical monitoring system 100. The monitoring system 100 includes a monitoring server 1, an intracardiac pressure value/waveform acquisition device 2, a biological signal measurement device 3, an in-hospital terminal 4, a gateway device 5, a biological signal measurement device 6, a user terminal 7, and a WiFi router 8. The monitoring server 1, the intracardiac pressure value/waveform acquisition device 2, the biological signal measurement device 3, the in-hospital terminal 4, and the gateway device 5 are installed in a medical facility such as a hospital and a clinic. The monitoring server 1, the intracardiac pressure value/waveform acquisition device 2, the biological signal measurement device 3, the in-hospital terminal 4, and the gateway device 5 are communicably connected by an in-hospital network LN. The biological signal measurement device 6, the user terminal 7, and the WiFi router 8 are installed in a living place such as a patient's home. The biological signal measurement device 6 and the user terminal 7 may be installed in a different location in the medical facility and connected to the in-hospital network LN. The monitoring server 1 may not necessarily be installed in a medical facility. Although two in-hospital terminals 4 and two user terminals 7 are described, one or three or more may be used.

For example, the monitoring server 1 is a server computer, a workstation, a personal computer (PC), and the like. In addition, the monitoring server 1 may include a plurality of computers, or may be a virtual machine virtually constructed by software or a quantum computer. Further, the function of the monitoring server 1 may be realized by a cloud service.

The intracardiac pressure value/waveform acquisition device 2 is not limited to a single device, and may be a combination of a plurality of devices. The intracardiac pressure value/waveform acquisition device 2 includes, for example, a catheter inspection device configured to measure intracardiac pressure values. The catheter to be used is a pigtail catheter, a balloon-equipped catheter, a Swan-Ganz catheter, a wedge pressure catheter, or the like. In addition, the intracardiac pressure value/waveform acquisition device 2 includes an ultrasonic diagnostic device or the like configured to measure and record an electrocardiogram.

In the present specification, a first measurement method performed by a medical worker involved in measurement is mainly a measurement method for obtaining some measured value by an invasive test, a test in which a medical worker entrusts a specimen acquired from a patient to a medical examination facility to perform analysis, a non-invasive test that cannot be performed only by a medical worker, or the like. The first measurement method performed by the medical worker involved also includes a measurement method for obtaining an evaluation value of a symptom by quantifying the degree of the symptom of the patient from an examination including interview, visual inspection, palpation, and auscultation by the medical worker. As a measurement method for quantifying the degree of the symptom of the patient from the examination to acquire the evaluation value of the symptom, for example, there is a method in which an appropriate numerical value is allocated to four classifications of a Nohria-Stevenson classification method which is one of the severity evaluation methods of heart failure, and the evaluation value of the symptom is quantified from the classification result to acquire the evaluation value of the symptom. In this case, the evaluation value is a feature amount (hereinafter also referred to as a feature value) and is a target variable. In addition, the first measurement method also includes a method in which a doctor determines which one of two or more predetermined stages corresponds to the degree of pulmonary congestion associated with heart failure based on results (i.e., medical images) obtained by chest X-ray examination, computed tomography (CT), and magnetic resonance imaging (MRI). In this case, each stage is indicated by a numerical value. The numerical value is a feature amount (or a feature value) and is a target variable. The measurement using the intracardiac pressure value/waveform acquisition device 2 corresponds to the first measurement method. The feature amount obtained from the first measurement method is data on which a target variable described below is based. Although the medical worker includes various types of occupations, in the present specification, in particular, a doctor, a nurse, a licensed practical nurse, a medical radiologist, a clinical laboratory technician, a hygiene laboratory technician, a clinical engineering technician, and a paramedic are assumed. The first measurement can also be said to be a measurement performed by a medical worker.

The biological signal measurement device 3 is an electrocardiogra capable of acquiring an electrocardiogram, a phonocardiograph capable of acquiring a phonocardiogram, and an electrocardiogramination device configured to acquire an electrocardiogram, a phonocardiogram, and a pulse wave. In addition, the biological signal measurement device 3 also includes a sphygmomanometer, a pulse wave meter, and the like. The measurement by the biological signal measurement device 3 includes a measurement that can be performed by a non-medical worker, but the measurement performed by the medical worker in the medical facility is the measurement by the first measurement method for convenience. The measurement by the first measurement method includes measurement of brain natriuretic peptide (BNP), NT-proBNP, uric acid level, inferior arterial diameter, ventricular ejection fraction, and central arterial pressure, and measurement of an evaluation value of symptoms.

The in-hospital terminal 4 is a terminal mainly used by a doctor or a nurse. The doctor and the nurse use the in-hospital terminal 4 to confirm the estimated value of the intracardiac pressure and the drug-taking status of the patient who has left the hospital and is living a daily life. The in-hospital terminal 4 is, for example, a desktop personal computer, a notebook personal computer, a tablet computer, a smartphone, or the like.

The gateway device 5 connects the in-hospital network LN and a global network GN such as the Internet. The gateway device 5 has a firewall function to block unauthorized access to the in-hospital network LN. A firewall device separate from the gateway device 5 may be installed.

Similarly to the biological signal measurement device 3, the biological signal measurement device 6 is an electrocardiograma phonocardiograph, an electrocardiogramamination device, a sphygmomanometer, and a pulse wave meter. The biological signal measurement device 6 is used by a patient at home. Therefore, it is desirable that the biological signal measurement device 6 be a device configured to be operated by a person who is not a medical worker such as the patient himself or herself or a family member thereof.

In the present specification, a second measurement method to be performed without requiring involvement of a medical worker in measurement is a method for measuring biological data of a patient mainly by non-invasive means. The second measurement method includes a method in which a patient obtains a measured value using a medical device in the patient's residence other than a medical facility by himself or herself or with help of their family. The measurement using the biological signal measurement device 6 is measurement by the second measurement method. The feature amount obtained from the second measurement method is data on which an explanatory variable described below is based. The second measurement can also be a measurement performed using a biological signal measurement device configured to be operated by a person who is not a medical worker.

The user terminal 7 is a terminal used by a patient. The user terminal 7 is, for example, a notebook computer, a tablet computer, a smartphone, or the like. The WiFi router 8 connects the user terminal 7 to the global network GN. The user terminal 7 receives the measurement result of the biological signal from the biological signal measurement device 6. The patient inputs the drug-taking status to the user terminal 7. The user terminal 7 transmits the measurement result of the biological signal and the drug-taking status to the monitoring server 1 via the WiFi router 8, the global network GN, or the like.

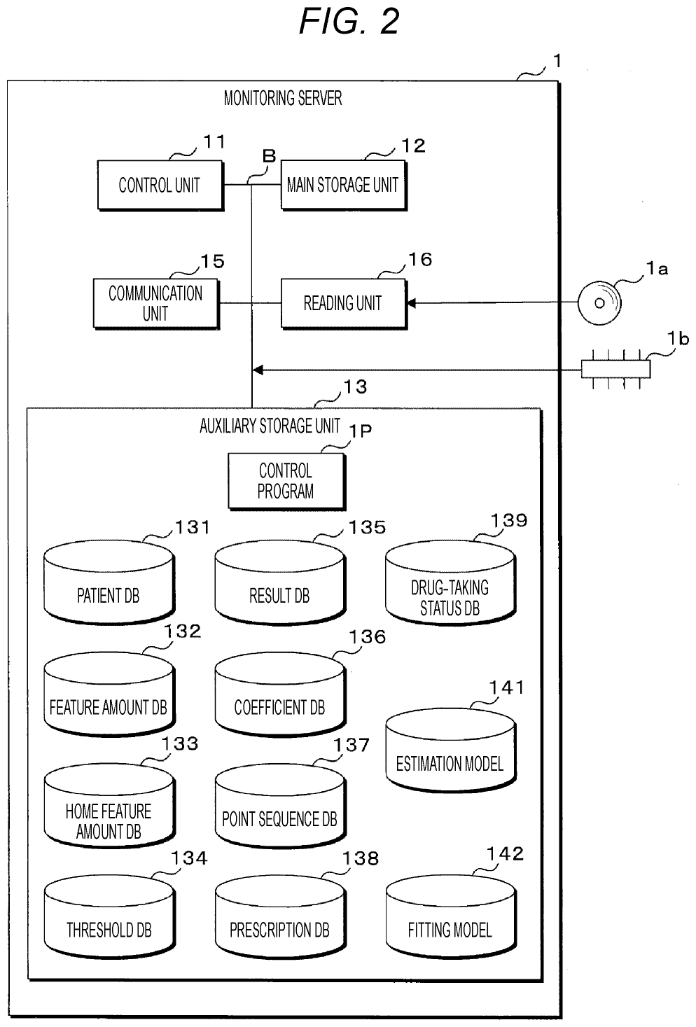

FIG. 2 is a block diagram illustrating a hardware configuration example of the monitoring server 1. The monitoring server 1 includes a control unit 11, a main storage unit 12, an auxiliary storage unit 13, a communication unit 15, and a reading unit 16. The control unit 11, the main storage unit 12, the auxiliary storage unit 13, the communication unit 15, and the reading unit 16 are connected by a bus B.

The control unit 11 includes one or a plurality of arithmetic processing devices such as a central processing unit (CPU), a micro-processing unit (MPU), and a graphics processing unit (GPU). The control unit 11 performs various types of information processing, control processing, and the like related to the monitoring server 1 by loading and executing a control program 1P stored in the auxiliary storage unit 13, and realizes functional units such as a first acquisition unit, a second acquisition unit, a third acquisition unit, a derivation unit, a generation unit, and an output unit.

The main storage unit 12 is a memory that includes a static random access memory (SRAM), a dynamic random access memory (DRAM), and a flash memory. The main storage unit 12 temporarily stores data necessary for the control unit 11 to execute arithmetic processing.

The auxiliary storage unit 13 is a hard disk, a solid state drive (SSD), or the like, and stores the control program 1P and various databases (DBs) necessary for the control unit 11 to execute processing. The auxiliary storage unit 13 stores a patient DB 131, a feature amount DB 132, a home feature amount DB 133, a threshold DB 134, a result DB 135, a coefficient DB 136, a point sequence DB 137, a prescription DB 138, and a drug-taking status DB 139. In addition, the auxiliary storage unit 13 stores one or more computer models including an estimation model 141 and a fitting model 142. The auxiliary storage unit 13 may be an external storage device externally connected separately from the monitoring server 1. Various DBs and the like stored in the auxiliary storage unit 13 may be stored in a database server or a cloud storage different from the monitoring server 1.

The communication unit 15 is a network interface circuit that communicates with the intracardiac pressure value/waveform acquisition device 2, the biological signal measurement device 3, the in-hospital terminal 4, and the gateway device 5 via the in-hospital network LN. The communication unit 15 communicates with the user terminal 7 via the gateway device 5, the global network GN, and the WiFi router 8. In addition, the control unit 11 may use the communication unit 15 to download the control program 1P from another computer via the global network GN or the like and store the control program 1P in the auxiliary storage unit 13.

The reading unit 16 reads a portable storage medium la including a compact disc (CD)-ROM or a digital versatile disc (DVD)-ROM. The control unit 11 may read the control program 1P from the portable storage medium la via the reading unit 16 and store the control program 1P in the auxiliary storage unit 13. Furthermore, the control unit 11 may read the control program 1P from a semiconductor memory 1b.

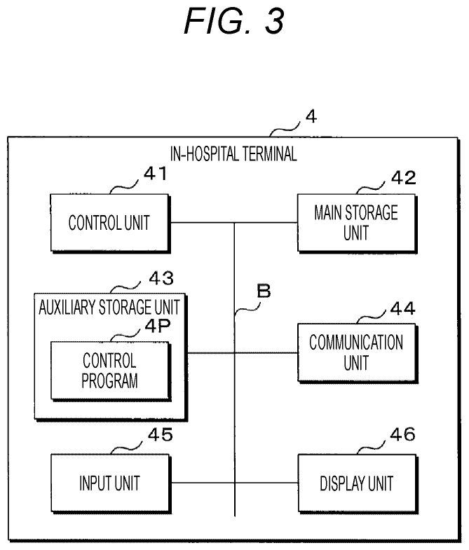

FIG. 3 is a block diagram illustrating a hardware configuration example of the in-hospital terminal 4. The in-hospital terminal 4 includes a control unit 41, a main storage unit 42, an auxiliary storage unit 43, a communication unit 44, an input unit 45, and a display unit 46. Those components are connected by a bus B.

The control unit 41 includes one or a plurality of arithmetic processing devices such as a CPU, an MPU, and a GPU. The control unit 41 provides various functions by reading and executing a control program 4P stored in the auxiliary storage unit 43

The main storage unit 42 is a memory including an SRAM, a DRAM, a flash memory, or the like. The main storage unit 42 temporarily stores data necessary for the control unit 41 to execute arithmetic processing.

The auxiliary storage unit 43 is a hard disk, an SSD, or the like, and stores various data necessary for the control unit 41 to execute processing. The auxiliary storage unit 43 may be an external storage device that is separate from the in-hospital terminal 4 and is externally connected. Various DBs and the like stored in the auxiliary storage unit 43 may be stored in a database server or a cloud storage.

The communication unit 44 communicates with the monitoring server 1 via the in-hospital network LN. In addition, the control unit 41 may use the communication unit 44 to download the control program 4P from another computer via the in-hospital network LN or the like and store the control program 4P in the auxiliary storage unit 43.

The input unit 45 is a keyboard or a mouse. The display unit 46 includes a liquid crystal display panel and the like. The display unit 46 displays the intracardiac pressure value and the like output by the monitoring server 1. Furthermore, the input unit 45 and the display unit 46 may be integrated into a touch panel display. Note that the in-hospital terminal 4 may perform display on an external display device.

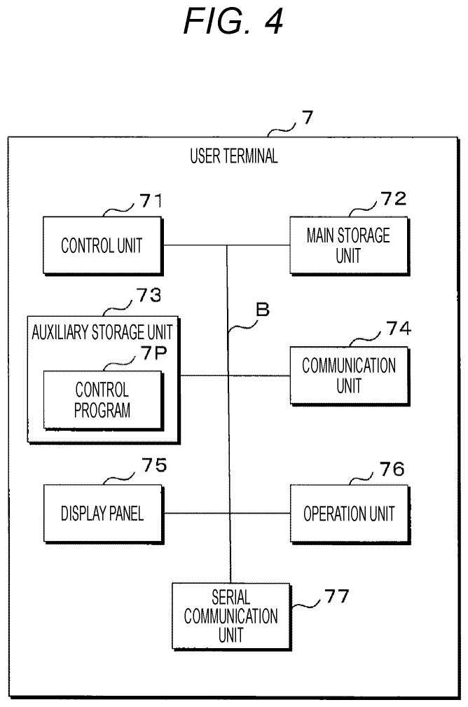

FIG. 4 is a block diagram illustrating a hardware configuration example of the user terminal 7. The user terminal 7 includes a control unit 71, a main storage unit 72, an auxiliary storage unit 73, a communication unit 74, a display panel 75, an operation unit 76, and a serial communication unit 77. Those components are connected by a bus B.

The control unit 71 includes one or a plurality of arithmetic processing devices such as a CPU, an MPU, and a GPU. The control unit 71 provides various functions by reading and executing a control program 7P stored in the auxiliary storage unit 73.

The main storage unit 72 is a memory that includes an SRAM, a DRAM, a flash memory, or the like. The main storage unit 72 temporarily stores data necessary for the control unit 71 to execute arithmetic processing.

The auxiliary storage unit 73 is a hard disk SSD, a memory card, or the like, and stores various data necessary for the control unit 71 to execute processing. The auxiliary storage unit 73 may be an external storage device externally connected separately from the user terminal 7. Various DBs and the like stored in the auxiliary storage unit 73 may be stored in a database server or a cloud storage.

The communication unit 74 communicates with the monitoring server 1 via the global network GN or the like. In addition, the control unit 71 may use the communication unit 74 to download the control program 7P from another computer via the global network GN or the like and store the control program 7P in the auxiliary storage unit 73.

The display panel 75 can include a liquid crystal panel, an organic electro luminescence (EL) display, or the like. The operation unit 76 can include, for example, a touch panel incorporated in the display panel 75, and a user can perform a predetermined operation performed on the display panel 75. In addition, the operation unit 76 can perform an operation on the software keyboard displayed on the display panel 75. Note that the operation unit 76 may be a hardware keyboard, a mouse, or the like.

The serial communication unit 77 is a communication interface that performs serial communication with another device. The serial communication unit 77 performs wired communication according to a universal serial bus (USB) standard and wireless communication according to a Bluetooth (registered trademark) standard or the like. The serial communication unit 77 receives waveform data and the like of the biological signal acquired by the biological signal measurement device 6.

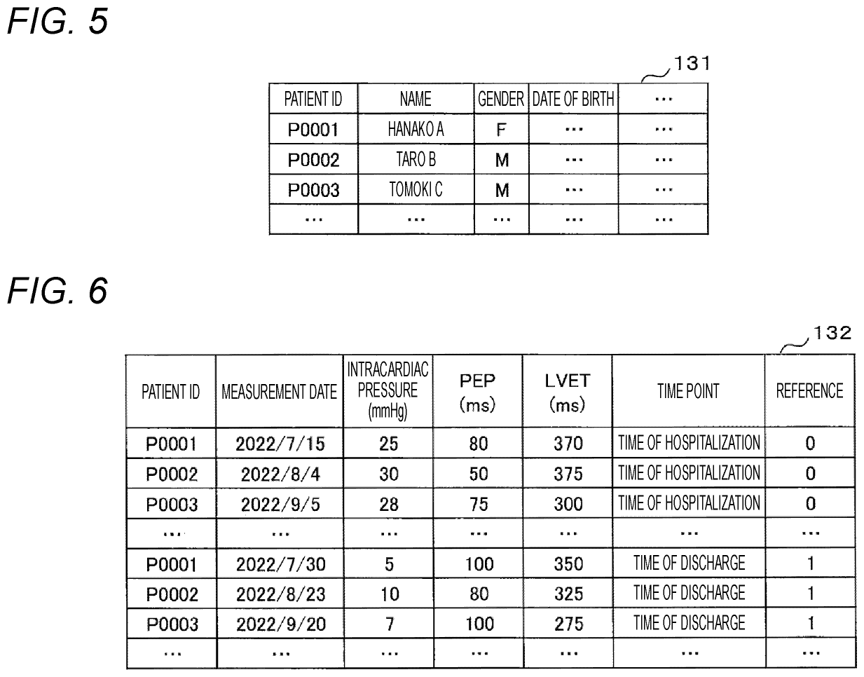

FIG. 5 is an explanatory diagram illustrating an example of the patient DB 131. The patient DB 131 stores patient information. The patient DB 131 stores a patient ID column, a name column, a gender column, and a date of birth column. The patient ID column stores a patient ID for identifying a patient. The patient ID may be a My Number (or an individual number) assigned to the patient by a government. The name column stores the name of the patient. The gender column stores the gender of the patient. For example, M represents a male, and F represents a female. The date of birth column stores the date of birth of the patient.

FIG. 6 is an explanatory diagram illustrating an example of the feature amount DB 132. The feature amount DB 132 stores patient feature amounts obtained from the intracardiac pressure value/waveform acquisition device 2 and the biological signal measurement device 3, or patient feature amounts obtained from waveforms, biological signals, or the like. The feature amount DB 132 includes a patient ID column, a measurement date column, an intracardiac pressure column, a PEP column, an LVET column, a time point column, and a reference column. The patient ID column stores a patient ID. The measurement date column stores the date when the feature amount, the waveform, the biological signal, or the like is measured. The intracardiac pressure column stores the intracardiac pressure. The unit is millimeters of mercury (mmHg).

More specifically, the intracardiac pressure is a systolic pressure, a diastolic pressure, or an average pressure of each part of the heart. Examples of the intracardiac pressure include right atrial pressure (systolic pressure, diastolic pressure, average pressure), right ventricular pressure (systolic pressure, diastolic pressure, end-diastolic pressure), pulmonary arterial pressure (systolic pressure, diastolic pressure, average pressure), left atrial pressure (systolic pressure, diastolic pressure, average pressure), left ventricular pressure (systolic pressure, diastolic pressure, end-diastolic pressure), and femoral arterial pressure. In the present embodiment, the intracardiac pressure column stores LVEDP (left ventricular end-diastolic pressure). The PEP column stores a pre-ejection period (PEP). The unit is milliseconds (ms). The LVET column stores the LVET (left ventricular ejection time). The time point column stores the time point at which the feature amount is obtained. At the time point, a word indicating the position of the patient in the course of the disease when the feature amount is obtained is stored. For example, the time point is the time of hospitalization, the time of discharge, or the like. The reference column stores whether to use the reference value for evaluating the change in the feature amount. It is indicated that the feature amount included in the record in which the reference column is 0 is not the reference value. It is indicated that the feature amount included in the record whose reference column is 1 is a reference value.

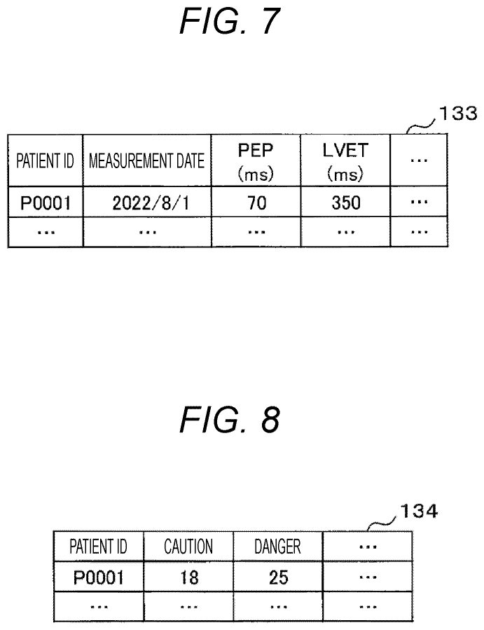

FIG. 7 is an explanatory diagram illustrating an example of the home feature amount DB 133. The home feature amount DB 133 stores a feature amount of a patient discharged from the hospital and monitored at home. The home feature amount DB 133 stores a feature amount of the patient obtained from the biological signal measurement device 6 or a feature amount of the patient obtained from a waveform, a biological signal, or the like. The home feature amount DB 133 includes a patient ID column, a measurement date column, a PEP column, and an LVET column. The patient ID column stores a patient ID. The measurement date column stores the feature amount or the date on which the waveform or the biological signal on which the feature amount is based is measured. The PEP column stores the pre-ejection period. The LVET column stores the left ventricular ejection time.

FIG. 8 is an explanatory diagram illustrating an example of the threshold DB 134. The threshold DB 134 stores, for each patient, a threshold used for determining the state of the patient from the intracardiac pressure. The threshold DB 134 includes a patient ID column, a caution column, and a danger column. The patient ID column stores a patient ID. The caution column stores a threshold for determining a state to be noted. The state to be noted is, for example, a congestive state requiring active intervention with a drug such as a diuretic, and a state requiring frequent monitoring by a medical worker. The danger column stores a another threshold for determining danger. The danger is, for example, an emergency state in which a symptom of heart failure exacerbation is appearing and treatment by a doctor is immediately necessary in a hospital, a state in which it is necessary to make a recommendation for a visit to a patient, a state in which it is necessary for a medical worker to visit, and the like.

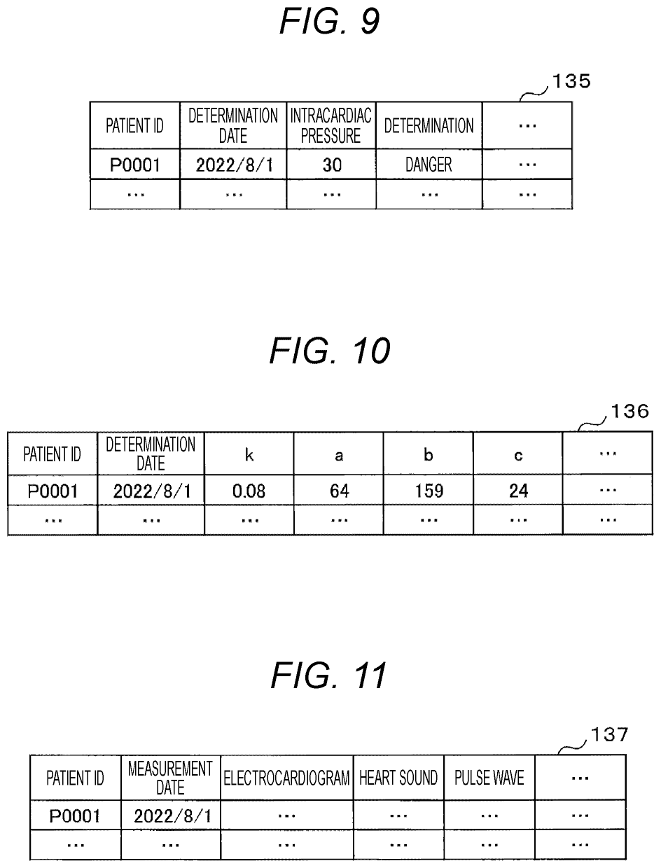

FIG. 9 is an explanatory diagram illustrating an example of the result DB 135. The result DB 135 stores a result of determination of the state or condition of a patient at home. The result DB 135 includes a patient ID column, a determination date column, an intracardiac pressure column, and a determination column. The patient ID column stores a patient ID. The determination date column stores the date on which the determination is made. The intracardiac pressure column stores the estimated intracardiac pressure. The determination column stores a determination result.

FIG. 10 is an explanatory diagram illustrating an example of the coefficient DB 136. The coefficient DB 136 stores a coefficient value of a function for curve-fitting the waveform of the intracardiac pressure. The model expression of the fitting function is, for example, Expression (1).

$$\begin{matrix}[ Math . 1 ] & \\ P ( t ) = \frac{a}{1 + b * {e}^{- k * t}} + c & ( 1 )\end{matrix}$$

The coefficient DB 136 includes a patient ID column, a determination date column, a k column, an a column, a b column, and a c column. The patient ID column stores a patient ID. The determination date column stores the date on which the determination is made. The k column, the a column, the b column, and the c column store the values of the coefficients k, a, b, and c of Expression (1), respectively.

FIG. 11 is an explanatory diagram illustrating an example of the point sequence DB 137. The point sequence DB 137 stores waveform data of the biological signal obtained from the biological signal measurement device 3 or the biological signal measurement device 6. The point sequence DB 137 includes a patient ID column, a measurement date column, an electrocardiogram column, a heart sound column, and a pulse wave column. The patient ID column stores a patient ID. The measurement date column stores a measured date. The electrocardiogram column stores waveform data of the electrocardiogram. The heart sound column stores phonocardiogram waveform data. The pulse wave column stores waveform data of the pulse wave. The waveform data is desirably stored in a general-purpose format. For example, the waveform data is in a format according to the medical waveform standardization description protocol managed by the MFER committee.

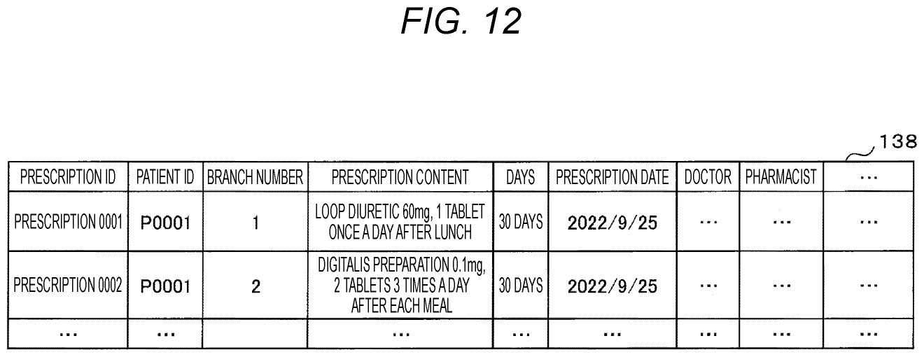

FIG. 12 is an explanatory diagram illustrating an example of the prescription DB 138. The prescription DB 138 stores information such as medicine prescribed by a doctor to a patient. The prescription DB 138 includes a prescription ID column, a patient ID column, a branch number column, a prescription content column, a days column, a prescription date column, a doctor column, and a pharmacist column. The prescription ID column stores a prescription ID specifying prescription. The prescription ID may be an ID given to the prescription. The patient ID column stores the patient ID of the patient prescribed the drug. In a case where a plurality of prescriptions, such as a case where a plurality of drugs is prescribed in one prescription, are included in the branch number column, the branch number for distinguishing each is stored. The prescription content column stores the contents of the prescription. The prescription content includes a drug name, an amount, and usage/dosage. The days column stores the number of prescribed days. The prescription date column stores the prescription date. The doctor column stores information of a doctor who has instructed prescription. The pharmacist column stores information of a pharmacist who has prescribed.

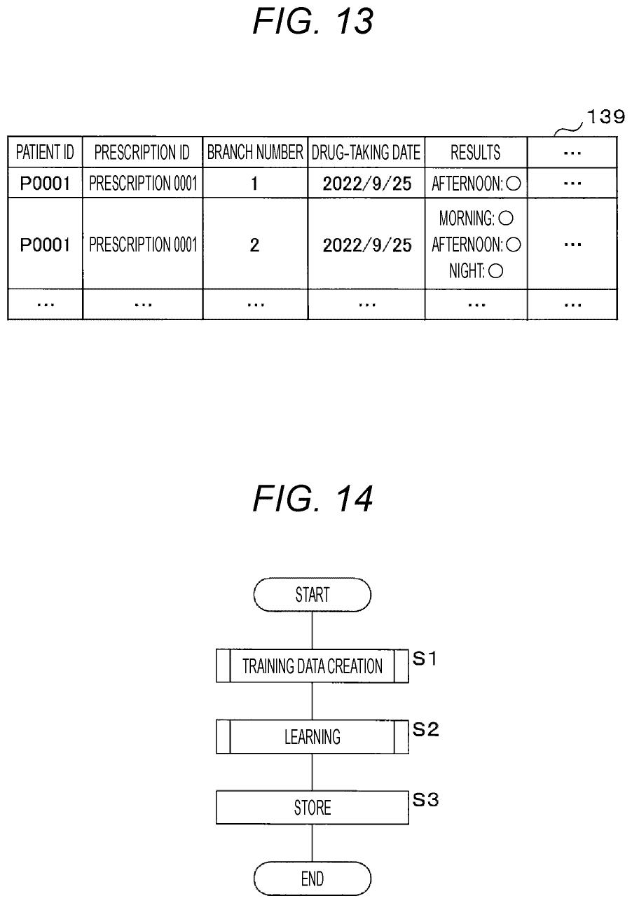

FIG. 13 is an explanatory diagram illustrating an example of the drug-taking status DB 139. The drug-taking status DB 139 stores the drug-taking status of the patient. The drug-taking status DB 139 includes a patient ID column, a prescription ID column, a branch number column, a dosing date column, and a result column. The patient ID column stores a patient ID. The prescription ID column stores a prescription ID. The branch number column stores a branch number. The dosing date column stores the date of each day of the dosing period. The result column stores a result of whether the patient has taken a drug. For example, o is stored in a case where the patient takes a drug, x is stored in a case where the patient does not take a drug, and the result column stores the result.

Next, information processing performed by the monitoring system 100 will be described.

FIG. 14 is a flowchart illustrating a procedure example of estimation model generation processing. The processing is a process of generating an estimation model 141. The control unit 11 of the monitoring server 1 creates training data (step S1). The control unit 11 performs machine learning using the training data (step S2). The control unit 11 stores the estimation model 141 obtained by machine learning (step S3), and ends the processing.

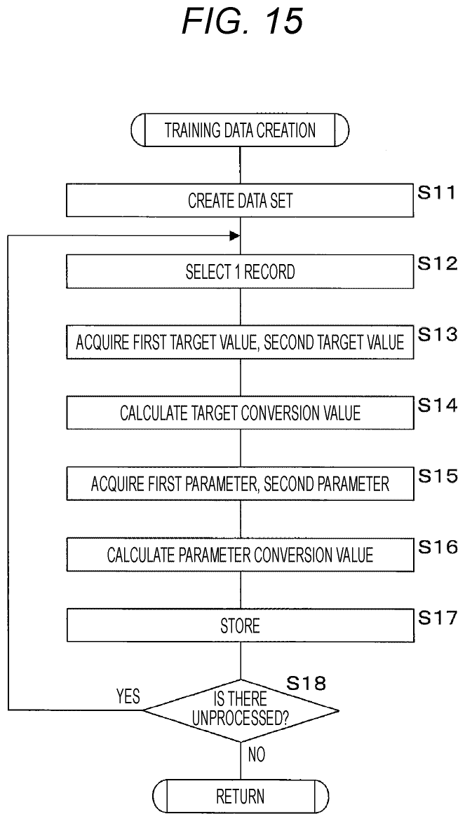

FIG. 15 is a flowchart illustrating a procedure example of the training data creation processing. The training data creation processing corresponds to step S1 in FIG. 14. The control unit 11 creates a data set (step S11). The data set is created from data of patients who have already been discharged from the hospital. A data set is obtained by combining a plurality of feature amounts obtained from catheter inspection data at the time of hospitalization (hereinafter referred to as the first time point) and discharge (hereinafter referred to as the second time point) of each patient, and a biological signal (e.g., electrocardiogram, heart sound, pulse wave, blood pressure) acquired on the same day as the day on which the catheter inspection data is acquired at each timing.

Here, the reason why the data at the time of hospitalization is used as the first time point and the data at the time of discharge is used as the second time point is that the patient's condition is considered to be extremely poor at the time of hospitalization, the patient's condition is considered to be recovered at the time of discharge, and the difference between the patient's conditions at the two time points can be said to be the most significant. As described above, by using data of both extreme states of the patient as a data set for training data creation, it is possible to obtain training data that can cover a wide measurement data range.

The control unit 11 acquires the feature amount from the feature amount DB 132. The feature amount obtained from the catheter inspection is LVEDP. The feature amounts obtained from the biological signal are the PEP and the LVET. The control unit 11 selects one record included in the data set as a processing target (step S12). The control unit 11 acquires a first target value and a second target value from the selected record (step S13). In the present embodiment, the first target value is LVEDP at the time of hospitalization (i.e., the first time point), and the second target value is LVEDP at the time of discharge (i.e., the second time point). The control unit 11 calculates a target conversion value (step S14). For example, a value obtained by dividing LVEDP (i.e., the first target value) at the time of hospitalization by LVEDP (i.e., the second target value) at the time of discharge based on data at the time of discharge is the target conversion value. A value obtained by subtracting the LVEDP at the time of discharge from the LVEDP at the time of hospitalization may be used as the target conversion value. The control unit 11 acquires a first parameter and a second parameter from the selected record (step S15). In the present embodiment, the first parameter is the PEP and the LVET at the time of hospitalization (i.e., the first time point), and the second target value is the PEP and the LVET at the time of discharge (i.e., the second time point). The control unit 11 calculates a parameter conversion value (step S16). For example, a value obtained by dividing the PEP and the LVET (hereinafter referred to as the first parameter) at the time of hospitalization by the PEP and the LVET (hereinafter referred to as the second parameter) at the time of discharge based on data at the time of discharge is the parameter conversion value. A value obtained by subtracting the PEP and the LVET (i.e., the second parameter) at the time of discharge from the PEP and the LVET (i.e., the first parameter) at the time of hospitalization may be used as the parameter conversion value. The control unit 11 stores the target conversion value and the parameter conversion value as training data in the auxiliary storage unit 13 (step S17). The control unit 11 determines whether there is an unprocessed record (step S18). In a case where it is determined that there is an unprocessed record (YES in step S18), the control unit 11 returns the processing to step S12 and performs processing on the unprocessed record. In a case where it is determined that there is no unprocessed record (NO in step S18), the control unit 11 returns the processing to the caller. Note that the reference time at the time of calculating the target conversion value and the parameter conversion value may be not the time of discharge but the time of hospitalization.

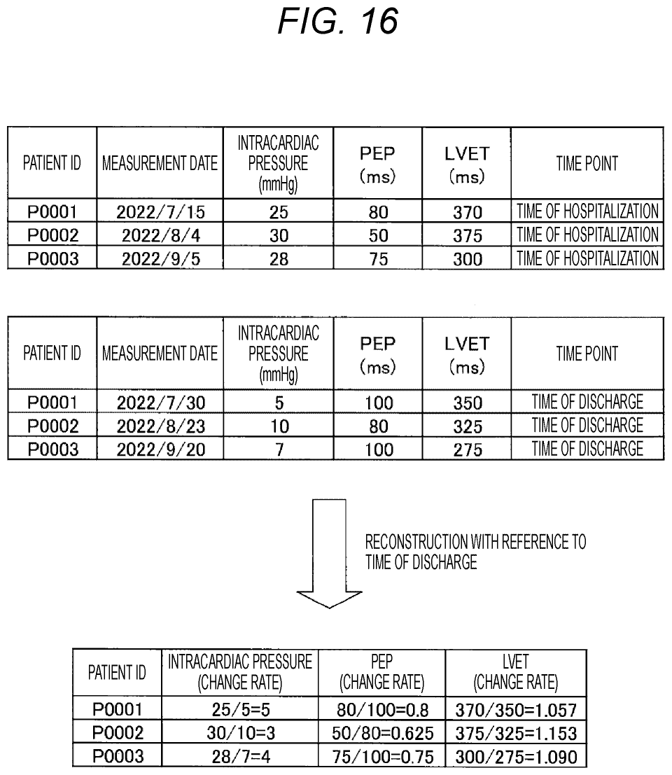

FIG. 16 is an explanatory diagram illustrating an example of constructing a data set. In FIG. 16, with reference to the data at the time of discharge, a value obtained by dividing the data at the time of hospitalization by the data at the time of discharge is used as reconstructed data. With reference to the data at the time of discharge, a value obtained by subtracting the data at the time of discharge from the data at the time of hospitalization is used as reconstructed data. With reference to the data at the time of hospitalization, a value obtained by dividing the data at the time of discharge by the data at the time of hospitalization or a value obtained by subtracting the data at the time of hospitalization from the data at the time of discharge may be used as reconstructed data.

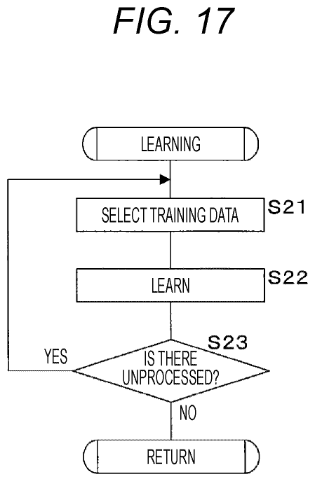

FIG. 17 is a flowchart illustrating a procedure example of the learning processing. The learning processing corresponds to step S2 in FIG. 14. The control unit 11 selects training data to be processed from among a plurality of pieces of training data created in the training data creation processing illustrated in FIG. 15 and stored in the auxiliary storage unit 13 (step S21). The control unit 11 performs learning based on the selected training data (step S22). The control unit 11 inputs the parameter conversion value, which is the explanatory variable and included in the training data to the estimation model 141, compares the value output from the estimation model 141 with the target conversion value, which is the target variable and included in the training data, and optimizes the parameter such as the weight between the neurons constituting the estimation model 141 so that the output value matches the target conversion value. The control unit 11 determines whether there is unprocessed training data (step S23). In a case where it is determined that there is unprocessed training data (YES in step S23), the control unit 11 returns the processing to step S21 and performs learning using the unprocessed training data. In a case where it is determined that there is no unprocessed training data (NO in step S23), the control unit 11 returns the processing to the caller.

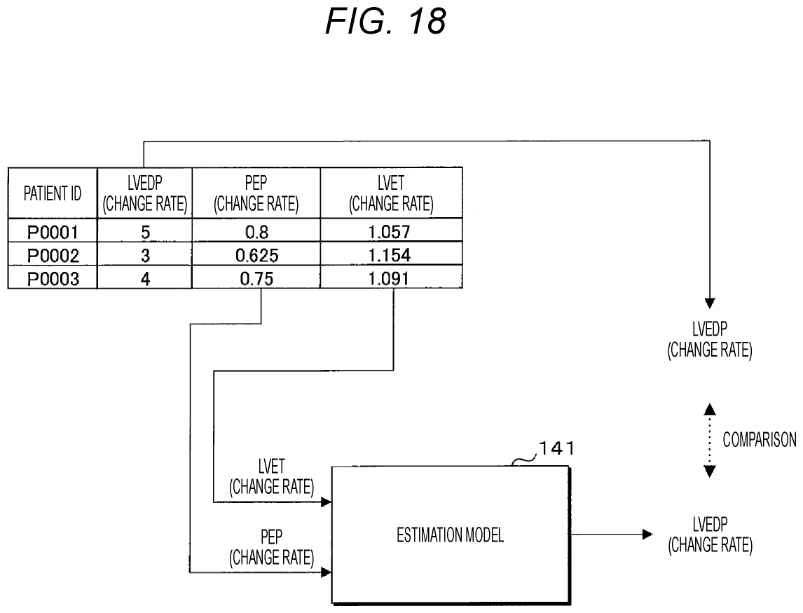

FIG. 18 is an explanatory diagram illustrating an example of an estimation model. The estimation model 141 is a neural network generated by deep learning using the above-described training data. The training data is created by the training data creation processing described above and stored in the auxiliary storage unit 13. The estimation model 141 is trained to output the target conversion value in a case where the parameter conversion value included in the training data is input. As described above, in the present embodiment, the parameter conversion value is a value obtained by dividing the first parameter (i.e., PEP and LVET at the time of hospitalization) by the second parameter (i.e., PEP and LVET at the time of discharge), and a change rate of the PEP and a change rate of the LVET. The target conversion value is a value obtained by dividing the first target value (i.e., LVEDP at the time of hospitalization) by the second target value (i.e., LVEDP at the time of discharge), and is a change rate of LVEDP.

In the generation processing of the estimation model 141, the change rate of the PEP and the change rate of the LVET included in the training data are input to the estimation model 141. By comparing the estimated value of the change rate of LVEDP from the estimation model 141 with the correct value of the change rate of LVEDP included in the training data, parameters such as weights between neurons constituting the estimation model 141 are optimized so that the output estimated value matches the correct value.

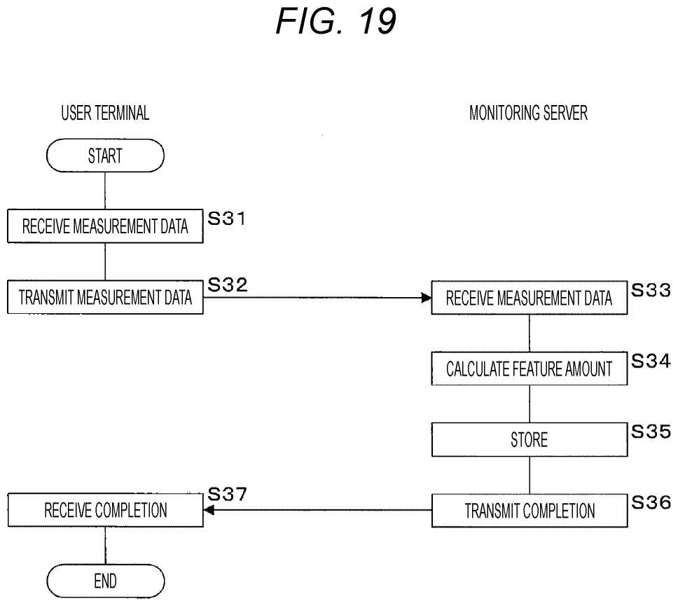

FIG. 19 is a flowchart illustrating a procedure example of the collection processing. The collection processing is a process of collecting measurement data such as a biological signal from a patient who has been discharged from the hospital. The patient who has been discharged from the hospital measures the electrocardiogram, heart sound, pulse wave, blood pressure, and the like by the biological signal measurement device 6 in the residence such as home, and transmits the electrocardiogram, heart sound, pulse wave, blood pressure, and the like to the user terminal 7. The control unit 71 of the user terminal 7 receives the measurement data from the biological signal measurement device 6 (step S31). Communication between the biological signal measurement device 6 and the user terminal 7 may be wireless communication such as WiFi or Bluetooth, or may be wired communication such as USB. In a case where communication cannot be performed, the biological signal measurement device 6 may write measurement data into a memory card, remove the memory card that has been written, and attach the memory card to the user terminal 7 to read the measurement data. Further, the biological signal measurement device 6 may display the measurement data as a two-dimensional code, image the two-dimensional code with the camera of the user terminal 7, and analyze the two-dimensional code to obtain the measurement data. The control unit 71 transmits the received measurement data to the monitoring server 1 (step S32). The control unit 11 of the monitoring server 1 receives the measurement data (step S33). The control unit 11 calculates a feature amount from the measurement data (step S34). The control unit 11 stores the feature amount in the home feature amount DB 133 (step S35). The control unit 11 transmits completion to the user terminal 7 (step S36). The control unit 71 of the user terminal 7 receives completion (step S37), and ends the processing. The feature amount stored in the home feature amount DB 133 is an example of a third parameter. The time point at which the biological signal measurement device 6 performs the measurement at the residence corresponds to a third time point.

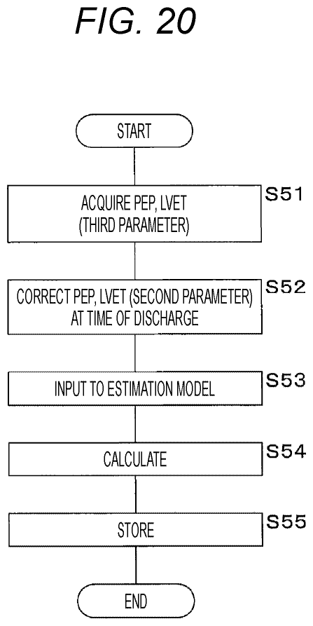

FIG. 20 is a flowchart illustrating a procedure example of the estimation processing. The estimation processing is a process of estimating the LVEDP using the PEP and the LVET obtained from the measurement data collected in the collection processing. The control unit 11 of the monitoring server 1 acquires the PEP and the LVET (i.e., the third parameter) from the home feature amount DB 133 (step S51). The control unit 11 corrects the acquired PEP and LVET with the reference values (i.e., the second parameters) (step S52). The reference values are the PEP and the LVET at the time of discharge. For example, the control unit 11 acquires the PEP and the LVET at the time of discharge for each patient from the feature amount DB 132, and divides the PEP and the LVET from the home feature amount DB 133 by the acquired PEP and LVET at the time of discharge. The control unit 11 inputs the corrected PEP and LVET (i.e., the parameter conversion value) to the estimation model 141 (step S53). The control unit 11 calculates an estimated value of the LVEDP (step S54). The control unit 11 calculates the estimated value by multiplying the reference value (i.e., LVEDP at the time of discharge) described above by the change rate (i.e., the target conversion value) output by the estimation model 141. The control unit 11 stores the calculated estimated LVEDP in the result DB 135 (step S55), and ends the processing. The control unit 11 repeats the number of times of the number of patients for which the home feature amount has been obtained and estimation processing.

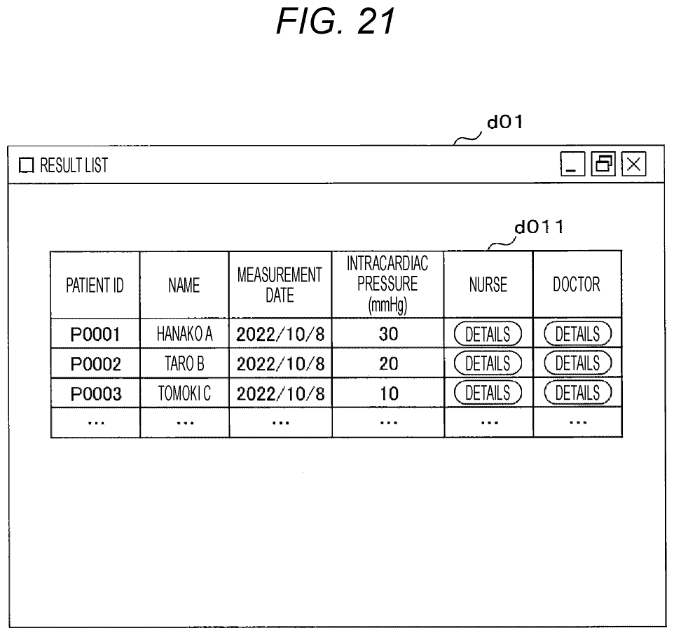

FIG. 21 is an explanatory diagram illustrating an example of a result list screen d01. The result list screen d01 is a screen displaying a list of the estimated intracardiac pressures. The result list screen d01 includes a list d011. The list d011 includes a patient ID column, a name column, a measurement date column, and an intracardiac pressure column. The list d011 may include a nurse column and a doctor column. The patient ID column displays a patient ID. The name column displays the name of the patient. The measurement date column displays the date on which the biological signal based on the feature amount has been measured. The intracardiac pressure column displays the estimated intracardiac pressure. Detail buttons are displayed in the nurse column and the doctor column. When the detail button is selected by mouse click or the like, a result screen for the selected patient is displayed. Although the patient's condition can be referred to on the result screen, it is desirable that the condition of each patient can be confirmed on the list screen. Since the condition can be classified into three situations of danger, attention, and normal on the basis of the threshold set for each patient, the display order of the patient in the result list screen is set to danger, attention, and normal. In addition, it is possible to determine whether the background color of the row, the color, the size, and the like of the value of the intracardiac pressure are dangerous, caution, or normal at a glance as different modes. Such a different display mode indicating that the patient's condition is in a danger situation or attention situation is an example of an alarm.

In the present embodiment, the following effects are obtained. It is possible to estimate the intracardiac pressure on the basis of the biological signal that can be measured even when the patient is at home. This makes it possible to remotely monitor whether there is an exacerbation of heart failure or a sign of exacerbation in a home patient. The training data used for generating the estimation model 141 is a reconstructed data set. Since the data set absorbs individual differences between patients, it is possible to generate the estimation model 141 with high accuracy.

In the above description, the estimation model 141 is not limited to a neural network. The estimation model 141 may be a computer model based on another learning algorithm such as a linear regression model, a decision tree, a random forest, a gradient boosting method, a support vector machine (SVM), or a nonlinear multiple regression method.

### Second Embodiment

The present embodiment relates to an aspect in which information other than an intracardiac pressure is also displayed on a screen so that a medical worker can more accurately grasp the condition of a home patient. In the following description, the same contents as those of the first embodiment will be omitted, and points different from those of the first embodiment will be mainly described.

The fitting model 142 for estimating a waveform indicating the temporal change of an intracardiac pressure will be described. The fitting model 142 is a machine learning model that estimates coefficients (k, a, b, and c) of a model expression (1) obtained by curve-fitting a left ventricular pressure waveform or a right ventricular pressure waveform indicating a temporal change in the left ventricular pressure or the right ventricular pressure. The fitting model 142 is trained to output a change rate of each coefficient of a model expression including a plurality of coefficients indicating a left ventricular pressure waveform or a right ventricular pressure waveform in a case where one or more values related to a heart rate or an arterial pressure are input.

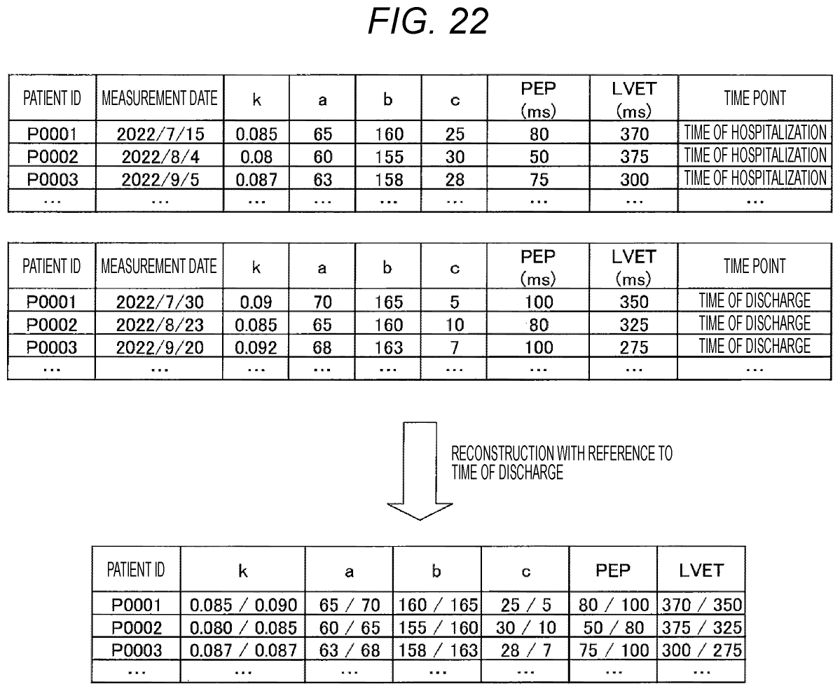

FIG. 22 is an explanatory diagram illustrating an example of constructing a data set. Similarly to the data set described with reference to FIG. 16, the data set is reconstructed so as to absorb individual differences among a plurality of patients. The reconstructed data is a value obtained by dividing data at the time of hospitalization by data at the time of discharge with reference to data at the time of discharge. With reference to the data at the time of discharge, a value obtained by subtracting the data at the time of discharge from the data at the time of hospitalization is used as reconstructed data. With reference to the data at the time of hospitalization, a value obtained by dividing the data at the time of discharge by the data at the time of hospitalization or a value obtained by subtracting the data at the time of hospitalization from the data at the time of discharge may be used as reconstructed data. The reason why the calculation is performed between two pieces of data with reference to either the data at the time of hospitalization or the data at the time of discharge is to absorb individual differences occurring between patients. Logarithmic conversion or the like may be used as long as individual differences can be absorbed. In addition, weighting may be performed for each data item.

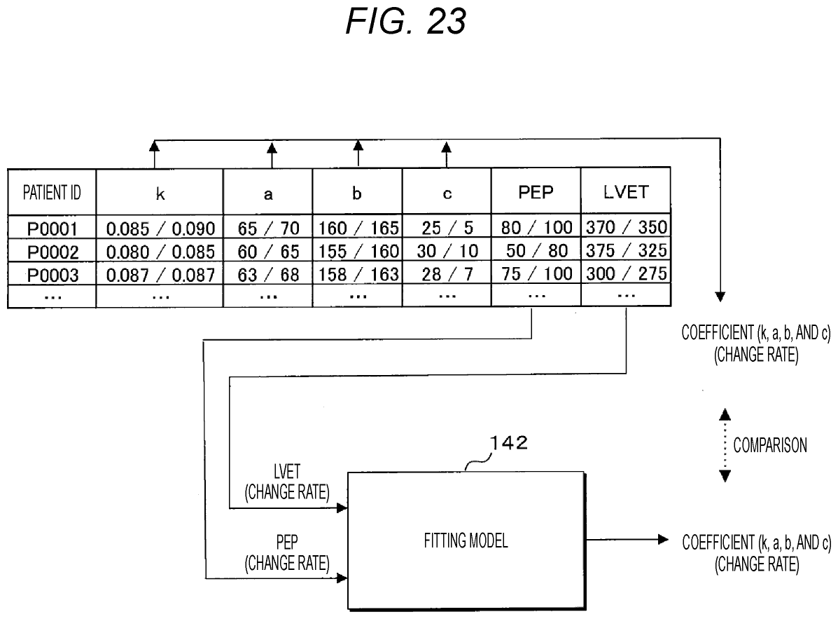

FIG. 23 is an explanatory diagram illustrating an example of a fitting model 142. The fitting model 142 is a neural network generated by deep learning using the data set illustrated in FIG. 22 as training data. The fitting model 142 is trained to output a change rate of a coefficient of a model expression indicating a left ventricular pressure waveform or a right ventricular pressure waveform in a case where one or more values related to a heart rate or an arterial pressure are input. In the present embodiment, inputs are the PEP and the LVET. The control unit 11 inputs the PEP and the LVET to the fitting model 142. The control unit 11 receives the change rate of the coefficients (k, a, b, and c) as the output of the fitting model 142. The control unit 11 can calculate the coefficients (k, a, b, and c) in the model expression (1) from the change rate of the coefficient and the reference value. The control unit 11 stores the calculated coefficient in the coefficient DB 136. Note that the fitting model 142 is not limited to the neural network, and may be a model based on another learning algorithm such as a linear regression model, a decision tree, a random forest, a gradient boosting method, a support vector machine (SVM), or a nonlinear multiple regression method.

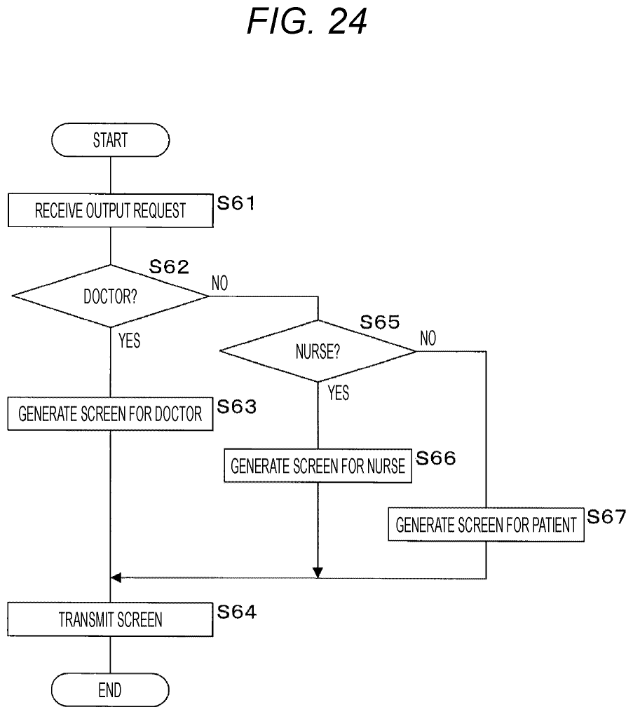

FIG. 24 is a flowchart illustrating a procedure example of result screen generation processing. The result screen generation processing is executed in a case where the detail button is selected on the result list screen d01 illustrated in FIG. 21. In addition, the result screen generation processing is executed in a case where there is a request from the user terminal 7. The control unit 41 of the in-hospital terminal 4 transmits an output request of the result screen to the monitoring server 1. The output request includes a patient ID for specifying a patient to be displayed and a screen type. In a case where the detail button in the nurse column is selected, a nurse is set as the screen type. In a case where the detail button in the doctor column is selected, the doctor is set as the screen type. The output request transmitted by the user terminal 7 includes a patient ID and a screen type. A patient is set as the screen type. The control unit 11 of the monitoring server 1 receives the output request (step S61). The control unit 11 determines whether the screen type included in the output request is a doctor (step S62). In a case where it is determined that the screen type is a doctor (YES in step S62), the control unit 11 generates a screen for a doctor (step S63). The control unit 11 transmits the generated screen to the in-hospital terminal 4 (step 64), and ends the processing.

In a case where it is determined that the screen type is not a doctor (NO in step S62), the control unit 11 determines whether the screen type is a nurse (step S65). In a case where it is determined that the screen type is a nurse (YES in step S65), the control unit 11 generates a screen for a nurse (step S66). The control unit 11 transmits the generated screen to the in-hospital terminal 4 (step S64), and ends the processing. In a case where it is determined that the screen type is not a nurse (NO in step S65), the control unit 11 generates a screen for a patient (step S67). The control unit 11 transmits the generated screen to the user terminal 7 (step S64), and ends the processing. Note that the determination of the screen type may be performed from the ID of the medical worker using the in-hospital terminal 4. For example, a medical worker database in which the ID of the medical worker and the job category (e.g., doctors, nurses, etc.) are associated with each other is stored in the auxiliary storage unit 13, and the job category can be determined from the ID.

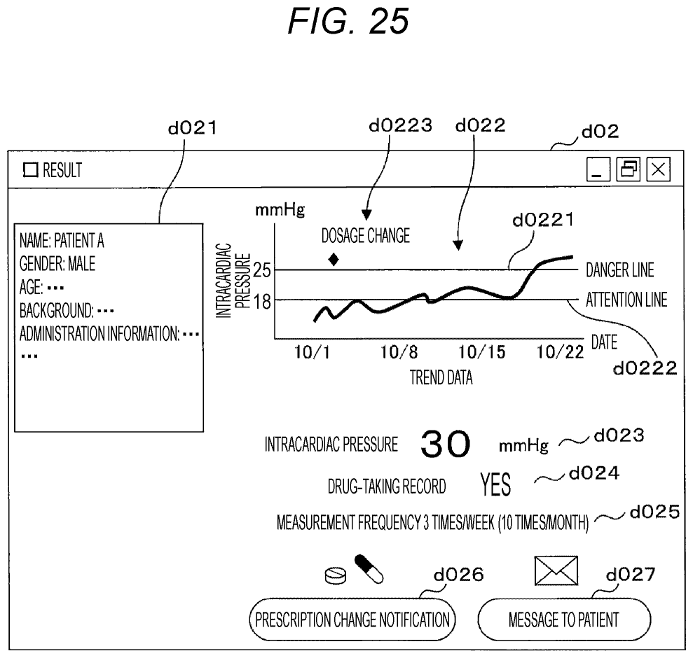

FIG. 25 is an explanatory diagram illustrating an example of a nurse result screen d02. A nurse result screen d02 includes a patient attribute d021, a trend graph d022, an intracardiac pressure d023, a drug-taking record status d024, a measurement frequency d025, a notification button d026, and a message button d027. The patient attribute d021 displays patient attributes such as the name, gender, and age of the patient. The trend graph d022 graphically displays the trend (i.e., time-series change) of the intracardiac pressure. The trend graph d022 includes a danger line d0221 and an attention line d0222. The trend graph d022 may include a dosage change indication d0223. The danger line d0221 is a line indicating a threshold for determining that the patient's condition is dangerous. The patient's dangerous condition refers to, for example, a condition in which the patient's condition is congestive requiring active intervention with a drug such as a diuretic, and frequent monitoring by a medical worker is necessary. The attention line d0222 is a line indicating a threshold for determining that the patient's condition requires attention. The attention to the patient's condition refers to, for example, a state in which it is necessary to make a visit recommendation to the patient in an emergency state in which the patient's condition is about to show symptoms of exacerbation and treatment by a doctor is immediately required in a hospital, a state in which it is necessary for a medical worker to visit, and the like. The danger line d0221 and the attention line d0222 may be shown in different manners (for example, different colors, different thicknesses, solid and dotted lines). Alternatively, the range below the danger line d0221 (the intracardiac pressure of 25 mmHg in FIG. 25), the range from the danger line d0221 (the intracardiac pressure of 25 mmHg in FIG. 25) to the attention line d0222 (18 mmHg in FIG. 25), and the range above the attention line d0222 (18 mmHg in FIG. 25) may be displayed in different manners (for example, fill with different colors, no fill and fill). As a result, it is possible to easily grasp whether the patient's condition is determined to be attention-needed or dangerous. Such different forms of display are examples of alarms. The dosage change indication d0223 is displayed at the position of the change date when the dosage of the drug is changed by the doctor during the display period. The dosage change indication d0223 is displayed by symbols as in FIG. 25, and may also display a graduation line or a descent line on the change date. This makes it possible to grasp the effect of treatment by changing the dosage. The intracardiac pressure d023 indicates an estimated value of the latest intracardiac pressure value. The drug-taking record status d024 indicates the presence or absence of a record that the patient has taken the drug. The control unit 11 generates the drug-taking record status d024 from the drug-taking status DB 139. By the drug-taking record status d024, it is possible to confirm whether the patient has forgotten to take the drug or has forgotten to record the drug-taking. The measurement frequency d025 displays a frequency at which the patient performs measurement by the biological signal measurement device 6 at home. By the measurement frequency d025, it is possible to confirm whether the patient has forgotten to perform measurement. In addition, the measurement frequency d025 is basic data for an insurance application. In a case where the estimated value of the intracardiac pressure value exceeds a predetermined threshold (i.e., danger line d0221 or attention line d0222), an alarm may be issued to the nurse by changing the frame of the screen, the title bar of the trend graph d022, the intracardiac pressure d023, and the color of the screen background. The notification button d026 is used to notify the patient that a prescription for a dosage change of a therapeutic agent for a circulatory disease such as a diuretic, a cardiotonic, or a vasodilator has been issued. The message button d027 is used to transmit a message to the patient. For example, in a case where the intracardiac pressure exceeds the attention line, a message recommending a visit to the hospital is transmitted.

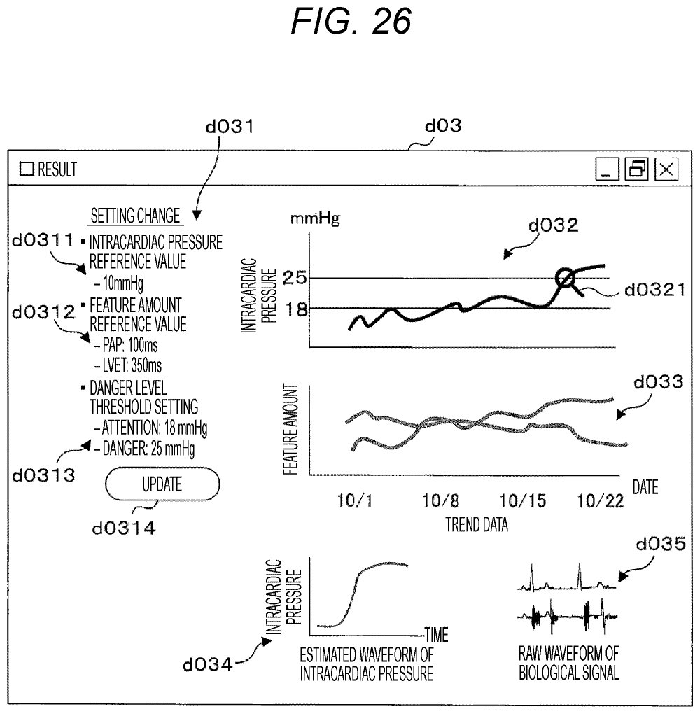

FIG. 26 is an explanatory diagram illustrating an example of a doctor result screen. A doctor result screen d03 includes a setting change region d031, an intracardiac pressure graph d032, a feature amount graph d033, an estimated waveform region d034, and a raw waveform d035 of the biological signal. The setting change region d031 is a region for changing settings related to estimation and evaluation of the intracardiac pressure. The setting change region d031 includes an intracardiac pressure reference value setting d0311, a feature amount reference value setting d0312, a danger level threshold setting d0313, and an update button d0314. The intracardiac pressure reference value setting d0311 displays a reference value of the intracardiac pressure. The feature amount reference value setting d03 12 displays a feature amount, here, a reference value between the PAP and the LVET. The danger level threshold setting d0313 displays a threshold of the intracardiac pressure for determining the patient's condition as being attentive and a threshold of the intracardiac pressure for determining the patient's condition as being dangerous. When the update button d0314 is selected by mouse click or the like, a screen for updating the intracardiac pressure reference value, the feature amount reference value, and the threshold is displayed. The doctor can change the intracardiac pressure reference value, the feature amount reference value, and the threshold using the screen. The intracardiac pressure graph d032 graphically displays the trend (i.e., time-series change) of the intracardiac pressure. When the mouse is over the graph, the mouse pointer becomes a pointer d0321 having a magnifying-glass shape, and the estimated waveform of the intracardiac pressure on the day indicated by the pointer d0321 is displayed in the estimated waveform region d034. The doctor can grasp the cardiac function of the patient by referring to the estimated waveform of the intracardiac pressure. The estimated waveform of the intracardiac pressure is a waveform drawn by the model expression (1). As described above, the coefficients (k, a, b, and c) of the model expression (1) is estimated using the fitting model 142. The feature amount graph d033 graphically displays the trend of the feature amount. The doctor refers to the trend of each feature amount as data when considering a treatment policy. The raw waveform d035 of the biological signal indicates the raw waveform of the biological signal on the day indicated by the pointer d0321. The control unit 11 displays the raw waveform using the point sequence data stored in the point sequence DB 137. By referring to the raw waveform, the doctor can check whether there is an abnormality that leads to deterioration of the patient's condition in each waveform. In a case where the estimated value of the intracardiac pressure value exceeds a predetermined threshold, an alarm may be issued to the doctor by changing the frame of the screen, the title bar of the intracardiac pressure graph d032, and the color of the screen background.

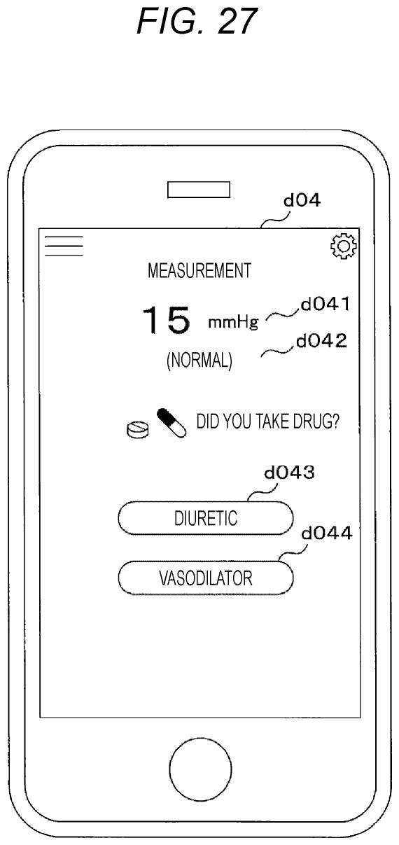

FIG. 27 is an explanatory diagram illustrating an example of a patient result screen d04. The patient result screen d04 includes an intracardiac pressure value d041, a determination result d042, a drug-taking button d043, and a drug-taking button d044. The intracardiac pressure value d041 is estimated using the estimation model 141. The determination result d042 is a determination result of the intracardiac pressure value. For example, there are three types of determination results of “normal”, “attention”, and “danger”. The drug-taking button d043 and the drug-taking button d044 are buttons for inputting a drug-taking history. In FIG. 27, when the patient selects the drug-taking button d043, the drug-taking history of the diuretic can be input, and when the patient selects the drug-taking button d044, the drug-taking history of the vasodilator can be input. In a case where only one type of drug is prescribed, only one drug button is displayed. In a case where three or more drugs are prescribed, the number of drug-taking buttons is the same as the number of types of drugs. The user terminal 7 transmits the input drug-taking history to the monitoring server 1.

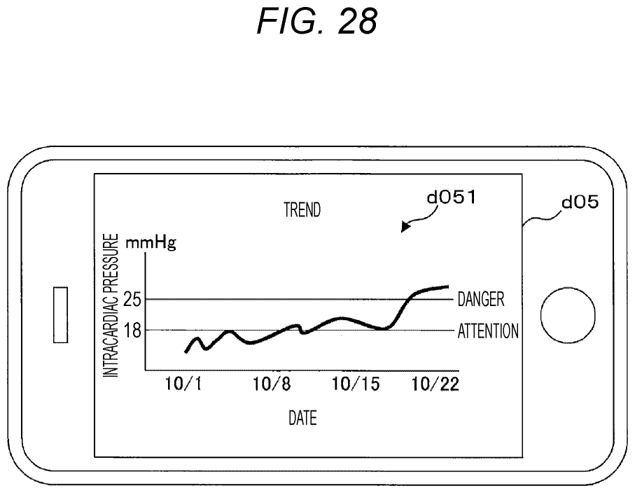

FIG. 28 is an explanatory diagram illustrating an example of a patient trend display screen d05. The trend display screen d05 includes a trend graph d051. The trend graph d051 is similar to the trend graph d022 illustrated in FIG. 25, and thus description thereof is omitted.

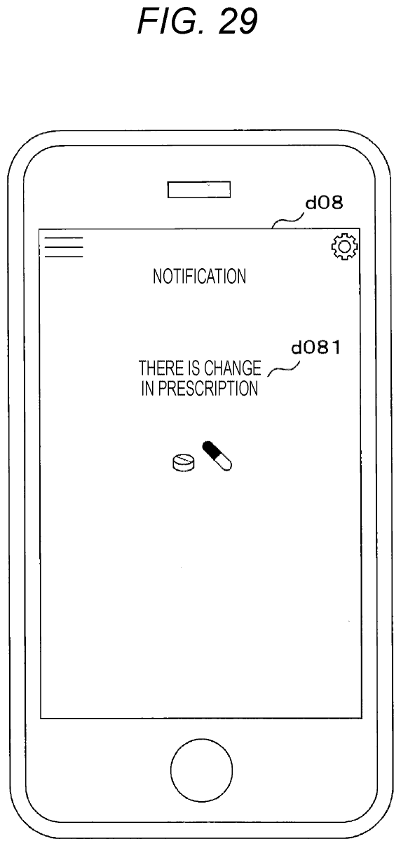

FIG. 29 is an explanatory diagram illustrating an example of a patient notification screen d08. The notification screen d08 includes a notification message d081. The notification message d081 is a message from the medical institution to the patient. The content of the message is, for example, a change in prescription or a recommendation of visit. As illustrated in FIG. 29, in a case where the message is the notification of the prescription change, the change content may be displayed by tapping the notification message d081. In a case where the estimated value of the intracardiac pressure value exceeds a predetermined threshold, the notification message d081 is issued as an alarm.

In the present embodiment, the following effects are obtained. Since the trend graph d022 of the nurse result screen d02 displays the danger line d0221 and the attention line d0222, it is possible to confirm the condition of the patient at a glance. From the dosage change indication d0223 of the trend graph d022 and the change in the trend graph d022, it is possible to grasp the effect of treatment by the dosage change. According to the drug-taking record status d024 of the nurse result screen d02, it is possible to confirm whether the patient has forgotten to take the drug or has forgotten to record the drug-taking. Furthermore, by referring to the drug-taking record status d024 and the trend graph d022, it can be used as a reference for determining whether the effect of drug is exhibited. By the measurement frequency d025 of the nurse result screen d02, it is possible to confirm whether the patient has forgotten to perform measurement. By the notification button d026 and the message button d027 of the nurse result screen d02, it is possible to call a screen for notifying the patient or creating a message.

By the setting change region d031 of the doctor result screen d03, the doctor can change the setting related to the estimation and evaluation of the intracardiac pressure. The doctor can accurately grasp the patient's condition from the intracardiac pressure graph d032 and the estimated waveform displayed in the estimated waveform region d034 of the doctor result screen d03. By referring to the feature amount graph d033 of the doctor result screen d03, the doctor can consider a future treatment policy. By referring to the raw waveform d035 of the doctor result screen d03, the doctor can check whether there is an abnormality that leads to deterioration of the patient's condition in each waveform.

By the intracardiac pressure value d041 and the determination result d042 of the patient result screen d04, the patient can confirm that the measurement has been performed and his/her condition. The drug-taking button on the patient result screen d04 enables the patient to check whether he/she has taken a drug and record the history of taking the drug.

The notification screen d08 enables notification and messages from the medical institution to the patient to be reliably transmitted. This makes it possible to raise attention when the patient forgets to take the drug. In addition, in a case where the deterioration tendency of the condition is sensed, it is possible to prevent the acute exacerbation in advance by making a patient's visiting recommendation and receiving an examination and an appropriate treatment.

In the above-described embodiments, the feature amount obtained by the first measurement method is LVEDP, but the present invention is not limited thereto. Intracardiac pressure, cardiovascular intracardiac pressure, and the like other than the LVEDP may be used as the feature amount. More specifically, the cardiovascular intracardiac pressure is a pressure or an average pressure of a blood vessel in the vicinity of the heart. The cardiovascular intracardiac pressure includes, for example, pulmonary artery wedge pressure (PAWP), pulmonary artery pressure (PAP), central venous pressure (CVP), and the like. Further, the pulmonary wedge pressure is also called a pulmonary arterial wedge pressure (PAWP), a pulmonary capillary wedge pressure (PCWP), or a pulmonary artery occlusion pressure (PAOP). In the above-described embodiments, the feature amount obtained by the second measurement method is the PEP and the LVET, but the present invention is not limited thereto. As the feature amount, the diastolic blood pressure, the systolic blood pressure, the maximum speed of rising of the pulse pressure waveform, the blood pressure value difference between the rising start point of the peripheral pulse pressure waveform and the dicrotic notch, the pulse wave increase coefficient, the heart rate, the isovolumetric systolic time, the pulse wave velocity, or the systolic time may be used.

The technical features or components described in the respective embodiments can be combined with each other, and new technical features can be formed by the combination. It should be understood that the embodiments disclosed herein are illustrative in all respects and are not restrictive. The scope of the present invention is defined not by the meanings described above but by the claims, and is intended to include meanings equivalent to the claims and all modifications within the scope. Some or all of the independent claims and their dependent claims described in the claims can be combined together, regardless of their dependent relationships. Although a form (multiple dependent claim form) in which a claim dependent on two or more other claims is described is used in the claims, the claim form is not limited thereto. The present invention may be described using a form in which a multiple dependent claim is dependent on at least one multiple dependent claim.

What is claimed is:

1. A method for training a machine learning model that is executed to assess a condition of a remotely monitored patient with heart failure, the method comprising:
generating training data by performing for each of a plurality of patients with heart failure:
acquiring at least a first target value based on a result of an invasive test performed at a first time and at least a first parameter value based on a result of a non-invasive test performed at the first time, and then acquiring at least a second target value based on a result of the invasive test performed at a second time and at least a second parameter value based on a result of the non-invasive test performed at the second time,
the first and second target values including at least one of: an intracardiac pressure, a value of brain natriuretic peptide (BNP), a value of N-terminal pro-B-type natriuretic peptide (NT-proBNP), a uric acid level, an inferior arterial diameter, and a ventricular ejection fraction, and
the first and second parameter values including at least one of: a pre-ejection period (PEP), a left ventricular ejection time (LVET), a diastolic blood pressure, a systolic blood pressure, a maximum rate of rise of a pulse pressure waveform, a pressure difference between a rising start point and a dicrotic notch of a peripheral pulse pressure waveform, a pulse wave increase coefficient, a heart rate, an isovolumetric systolic time, a pulse wave velocity, and a systolic time,
deriving a target value change ratio based on the first and second target values,
deriving a parameter value change ratio based on the first and second parameter values, and
storing the target value change ratio in association with the parameter value change ratio as the training data; and
training a machine learning model with the generated training data such that a target value change ratio is generated in response to an input of an actual parameter value change ratio derived for a remotely monitored patient with heart failure.

2. The method according to claim 1, further comprising:
performing the invasive and non-invasive test at the first time when the patient is in a hospital; and
performing the invasive and non-invasive test at the second time when the patient is discharged from the hospital.

3. The method according to claim 1, wherein
performing the invasive test includes using a catheter inspection device.

4. The method according to claim 3, wherein
performing the non-invasive test includes using an electrocardiogra phonocardiograph, an electrocardiogramamination device, a sphygmomanometer, or a pulse wave meter.

5. The method according to claim 1, wherein
the machine learning model is a deep-learning neural network.

6. A monitoring device for remotely monitoring a condition of a patient with heart failure, comprising:
an interface circuit connectable to a display device;
a memory that stores a program; and
a processor configured to execute the program to:
acquire at least a first target value based on a result of an invasive test performed on the patient at a first time and at least a first parameter value based on a result of a non-invasive test performed on the patient at the first time, and then acquire at least a second parameter value based on a result of the non-invasive test performed on the patient at a second time,
the first target value including at least one of: an intracardiac pressure, a value of brain natriuretic peptide (BNP), a value of N-terminal pro-B-type natriuretic peptide (NT-proBNP), a uric acid level, an inferior arterial diameter, and a ventricular ejection fraction,
the first and second parameters including at least one of: a pre-ejection period (PEP), a left ventricular ejection time (LVET), a diastolic blood pressure, a systolic blood pressure, a maximum rate of rise of a pulse pressure waveform, a pressure difference between a rising start point and a dicrotic notch of a peripheral pulse pressure waveform, a pulse wave increase coefficient, a heart rate, an isovolumetric systolic time, a pulse wave velocity, and a systolic time,
derive a parameter value change ratio based on the first and second parameter values,
execute a call to a machine learning model with the parameter value change ratio to determine a target value change ratio, the machine learning model having been trained with target value change ratios and parameter value change ratios that are derived from results of the invasive and non-invasive tests performed on patients with heart failure and are associated with each other,
convert the first target value into a second target value corresponding to the second time using the target value change ratio that is output from the machine learning model, and
transmit the second target value to the display device for the second target value to be displayed on the display device for heart condition monitoring.

7. The monitoring device according to claim 6, wherein
the processor is configured to execute the program to acquire the first target value from a catheter inspection device, and acquire the first and second parameter values from a biological signal measurement device.

8. The monitoring device according to claim 6, wherein
the second time is at least one day later than the first time.

9. The monitoring device according to claim 6, wherein
the processor is configured to execute the program to cause the display device to display a graph of the first and second target values in time series.

10. The monitoring device according to claim 9, wherein
the processor is configured to execute the program to cause the display device to display, on the graph, a mark indicating when an amount of drug administered to the patient is changed.

11. The monitoring device according to claim 6, wherein
the processor is configured to execute the program to:
determine whether the second target value exceeds a threshold, and
upon determining that the second target value exceeds the threshold, cause the display device to output an alert.

12. The monitoring device according to claim 11, wherein
the processor is configured to execute the program to modify the threshold upon receipt of a request from the display device.

13. The monitoring device according to claim 6, wherein
the processor is configured to execute the program to cause the display device to display a graph of the first and second parameters in time series.

14. The monitoring device according to claim 6, wherein
the processor is configured to execute the program to:
acquire a drug-taking record from a terminal operated by the patient, and
cause the display device to display the drug-taking record together with the first and second target values.

15. A monitoring method for remotely monitoring a condition of a patient with heart failure, the method comprising:
acquiring at least a first target value based on a result of an invasive test performed on the patient at a first time and at least a first parameter value based on a result of a non-invasive test performed on the patient at the first time, and then acquiring at least a second parameter value based on a result of the non-invasive test performed on the patient at a second time,
the first target value including at least one of: an intracardiac pressure, a value of brain natriuretic peptide (BNP), a value of N-terminal pro-B-type natriuretic peptide (NT-proBNP), a uric acid level, an inferior arterial diameter, and a ventricular ejection fraction,
the first and second parameters including at least one of: a pre-ejection period (PEP), a left ventricular ejection time (LVET), a diastolic blood pressure, a systolic blood pressure, a maximum rate of rise of a pulse pressure waveform, a pressure difference between a rising start point and a dicrotic notch of a peripheral pulse pressure waveform, a pulse wave increase coefficient, a heart rate, an isovolumetric systolic time, a pulse wave velocity, and a systolic time;
deriving a parameter value change ratio based on the first and second parameter values;
executing a call to a machine learning model with the parameter value change ratio to determine a target value change ratio, the machine learning model having been trained with target value change ratios and parameter value change ratios that are derived from results of the invasive and non-invasive tests performed on patients with heart failure and are associated with each other;
converting the first target value into a second target value corresponding to the second time using the target value change ratio that is determined using the machine learning model; and
displaying the second target value for heart condition monitoring.

16. The monitoring method according to claim 15, wherein
the first target value is acquired from a catheter inspection device, and the first and second parameter values are acquired from a biological signal measurement device.

17. The monitoring method according to claim 15, wherein
the second time is at least one day later than the first time.

18. The monitoring method according to claim 15, further comprising:
displaying a graph of the first and second target values in time series.

19. The monitoring method according to claim 18, further comprising:
displaying, on the graph, a mark indicating when an amount of drug administered to the patient is changed.

20. The monitoring method according to claim 15, further comprising:
determining whether the second target value exceeds a threshold; and
upon determining that the second target value exceeds the threshold, outputting an alert.
